(19) United States
(12) Patent Application Publication
(10) Pub. No.: US 2010/0291105 A1
(43) Pub. Date: Nov. 18, 2010

(54) AGENT FOR THE TREATMENT OF MALIGNANT DISEASES

(76) Inventors: Elke Pogge Von Strandmann, Koln (DE); Andreas Engert, Koln (DE)

Correspondence Address: BERLINER & ASSOCIATES, 555 WEST FIFTH STREET, 31ST FLOOR, LOS ANGELES, CA, 90013, US

(21) Appl. No.: 12/594,787

(22) PCT Filed: May 3, 2007
(86) PCT No.: PCT/EP07/03901

§ 371 Date: Jun. 29, 2010

(30) Foreign Application Priority Data
May 3, 2006 (DE) 10 2006 020 317.8
Jun. 21, 2006 (DE) 10 2006 028 893.9

(51) Int. Cl. A61K 39/395; A61K 38/17; C07K 14/435; C07K 16/18; C07H 21/00; G01N 33/566; C12N 5/10; A61P 35/00

(52) U.S. Cl. 424/172.1; 514/19.2; 530/350; 530/389.1; 536/23.4; 435/7.23; 435/325

(57) ABSTRACT

The invention proposes an agent for the treatment of tumor diseases, for example the multiple myeloma, said agent acting upon NK cells through the activation of an anti-tumor immune response after stimulation of the NKp30 receptor and the natural cytotoxicity receptors (NCR). Said agent contains in an acceptable carrier material a physiologically effective amount of the BAT3 protein and/or BAT3/antiCD138 or BAT3-specific antibodies or derivatives of these substances. According to the principle identical to that adopted for the BAT3/anti-CD138 protein the invention may also be used for the treatment of CD138-negative tumors. For this purpose the antiCD138 component with an antibody fragment is replaced by an optional tumor antigen and the respective agent is used for the therapy of tumors expressing this tumor antigen. Moreover, the invention also proposes the use of recombinant BAT3 protein or a BAT3 fragment without anti-body-based fusion component for the treatment of malignant diseases by activating NKp30 and NCR on NK cells. Finally, the invention relates to the use of BAT3 cDNA for in-vivo and/or ex-vivo introduction of BAT3 into tumor cells leading to an improved detection by NK cells in the immunotherapy of malignant diseases.

The invention relates to an agent for the treatment of tumor diseases, autoimmune diseases or for use in the cellular immune therapy in the framework of allogeneic and autologous transplants by activating or inhibiting of BAT3 with a physiologically effective amount of the BAT3 protein (HLA-associated transcript 3) and/or BAT3 in connection with a tumor-specific antibody fragment of the specifically encoded DNA (cDNA) and/or RNA for the production of such an agent in the event of an activation of the BAT3 related disease. The invention furthermore relates to the use of the BAT3 cDNA for the ex vivo and in vivo overexpression of BAT3 in tumor cells after viral or non-viral gene transfer with a view to elicit an anti-tumor immune response in tumor patients. Moreover, it relates to an agent for the inhibition or activation of BAT3 by antagonistic or agonistic antibodies, antisense nucleotides or specific inhibitors. Moreover, it relates to a diagnostic marker such as, for example, a specific antibody for diagnosis of the disease and clinical monitoring/follow-up.

### DESCRIPTION

The invention relates to an agent for the treatment of diseases in humans by means of the recombinant protein BAT3, the encoding DNA/RNA, an inhibitor/activator of BAT3 in the form of antibodies or antisense nucleotides or other specific inhibitors and by means of BAT3 synthesized via a suitable linker in conjunction with a single chain antibody fragment of the mouse monoclonal antibody B-B4 to the tumor-specific CD138 antigen.

Additionally, the invention relates to the use of the BAT3 cDNA for the ex vivo and in vivo overexpression of BAT3 in human tumor cells after viral or non-viral gene transfer with a view to bring about an NKp30 induced immune response to act on the tumor cells encountered with malignant diseases of man.

Being part of the innate immune system NK cells which are capable of detecting and directly attacking malignant cells and contributing to the formation of an adaptive immune response to tumor cells, have in recent times been increasingly used for the immune therapy of malignant diseases.

The activity of NK cells to counteract tumor cells is controlled via activating receptors among which are the natural cytotoxicity receptors (NCR) including NKp30 as an important member of this family. The stimulation of the receptors as well as of NKp30 is based on the interaction with the corresponding ligands on malignant cells and leads to lysis of the target cells (D. Pende et al., J Exp Med 190, 1505 (Nov. 15, 1999)). Therefore, the ligand (or possibly the ligands) of NKp30 is basically suited for the therapy of tumors. However, the cellular ligand has only been described indirectly hitherto with the aid of masking antibodies while the molecular identification of the ligand had been pending (L. Moretta, A. Moretta, Embo J 23, 255 (Jan. 28, 2004)).

Contrary to expectations that a ligand of NKp30 is a membrane protein of tumor cells we have identified a nuclear protein as cellular ligand of the NKp30 receptor. In the enclosed manuscript we have described the cloning of BAT3, the first cellular tumor-associated ligand of the surface receptor NKp30, an important activating immune receptor of NK cells (L. Moretta, A. Moretta, Embo J 23, 255 (Jan. 28, 2004)). Surprisingly, this nuclear protein reaches the surface of the cells and, moreover, is discharged from the cells as soon as the cells receive a “stress signal”. This may be a heat shock or contact with NK cells.

Depending on context, BAT3 may lead to inhibition or activation of NK cells which primarily is mediated by the natural cytotoxicity receptors (NCR).

BAT3 may thus cause an activation (cytotoxicity, cytokine secretion) on the cell surface or associated with exosomes in the supernatant of tumor cells. However, the recombinant, purified protein inhibits the activity of NK cells. On the other hand, the addition of BAT3 specific, cross-linking antibodies in combination with purified BAT3 leads to a dramatic NK cell activity increase.

All in all our data provide elucidation to the effect that the immobilized form of BAT3 whether on the cell surface, on exosomes or through antibodies causes an NK activation whereas the soluble form leads to inhibition. This soluble form may, for example, develop through shedding of the molecule and may thus be viewed as a mechanism of tumor and virus-infected cells to avoid detection by the immune system. This interpretation is supported by evidence that BAT3 increases significantly in the serum of tumor patients in comparison to healthy donors (Example Hodgkin's lymphoma—Comparison between 40 patient sera and 40 normal sera).

This is a mechanism hitherto unknown or never described in any hypothesis via which important surface receptors of NK cells (NKp30, NCR) are controlled by a nuclear protein (BAT3), with the translocalization of BAT3 to the cell surface and cell environment being inducible.

With regard to all relevant structural data reference is made to pertinent publications and papers/contributions cited therein; (NKp30: D. Pende et al., J Exp Med 190, 1505 (Nov. 15, 1999); L. Moretta, A. Moretta, Embo J 23, 255 (Jan. 28, 2004); and BAT3: J. Banerji, J. Sands, J. L. Strominger, T. Spies, Proc Natl Acad Sci USA 87, 2374 (Mar., 1990)).

It is known that the expression of the NKp30 specific ligands to tumor cells correlates directly with their sensibility towards NK cells so that the loss of ligands is to be seen as a strategy of the tumor cells aimed at escaping control by the immune system.

However, this does not only apply to NKp30 but also to the already known ligands of a second activating receptor of NK cells, the NKG2D receptor. In this case gene-therapeutic preclinical data have already been elaborated showing that an overexpression of NKG2D ligands results in the immune system successfully combating the relevant tumor. This activation is not dependent on the activity of the inhibiting NK receptors which normally prevent NK cells from attacking the body's own cells (e.g. cf. A. Cerwenka, J. L. Baron, L. L. Lanier, Proc Natl Aced Sci USA 98, 11521 (Sep. 25, 2001); A. Diefenbach, E. R. Jensen, A. M. Jamieson, D. H. Raulet, Nature 413, 165 (Sep. 13, 2001)).

NKG2D-ligand based recombinant constructs actually cause the promising anti-tumoral activity as has been pointed out in various preclinical studies (C. Germain et al, Clin Cancer Res 11, 7516 (Oct. 15, 2005); E. Pogge von Strandmann et al., Blood (Oct. 6, 2005)).

Our identification of BAT3 as cellular ligands of NKp30 and our investigations relating to the functions of BAT3 have shown that this new BAT3 ligand is suited for a tumor therapy aimed at a NKp30/NCR mediated stimulation of NK cells.

Our investigations have shown that the overexpression of BAT3 in tumor cells after induction through a heat shock or contact with NK cells causes BAT3 to be released and leads to a measurable activation of NK cells. Likewise, human tumor cell lines such as 293T and RPMI cells secrete endogenous BAT3, whereas colon carcinoma lines such as LS175T are incapable of doing this (see enclosed manuscript). It is to be assumed that BAT3 even without targeting is functional on tumor cells via an antibody fragment or via an antibody and may lead to NK cell activation.

Object of the invention is therefore to propose an agent for the therapy of patients suffering from malignant diseases, said agent containing a physiologically effective amount of the protein BAT3 as active substance in a pharmaceutically acceptable carrier material.

Therapies that are suited to trigger an anti-tumor immune response of patients are in great demand. This also holds good for therapies involving transplants or aimed at diseases stemming from the immunologic spectrum which have not yet reached maturity at all. During recent years a multitude of immunotherapeutic strategies have been developed, or are currently under clinical evaluation, which are primarily based on the use of antibodies.

An example for the application of BAT3 in tumor therapies is its use as fusion protein with an antibody fragment which enables a specific bonding to tumor cells. For example, a BAT3/anti-CD138 shall be used therapeutically to support an anti-tumor immune response of patients suffering from multiple myeloma, similar to what has been shown for the bispecific protein ULBP2/CD138 (Pogge et al., 2006). Basically, the B-B4 single chain antibody fragment used with specificity for CD138 (Syndecan 1) is very well suited for this application. CD138 is a member of the Syndecan family, heparan sulfate proteoglycans, playing a role in cell adhesion, differentiation and proliferation, and often is overexpressed on malignant multiple myeloma cells whereas it is missing on other hemotopoetic cells (Dhodapkar MV, Sanderson RD. et al., Leuk Lymphoma. 1999;34:35-43).

Moreover, no satisfactory therapy option has been available for the treatment of multiple myeloma. The life expectancy of patients suffering from it is merely a few months after first diagnosis.

Another example is the inhibition of BAT3 with autoimmune diseases to modulate an over-reactive immune system.

Antagonistic and agonistic BAT3-specific antibodies and recombinantly produced BAT3 protein may be used for the therapy of malignant diseases and immunologic diseases.

An activation of NK cells in the framework of cellular immunotherapy with the help of recombinant BAT3 or derivatives/antibodies within the culturing phase of these cells prior to transplant but also as a systemic step after transplant is to be regarded as yet another novel approach improving therapeutic endeavors.

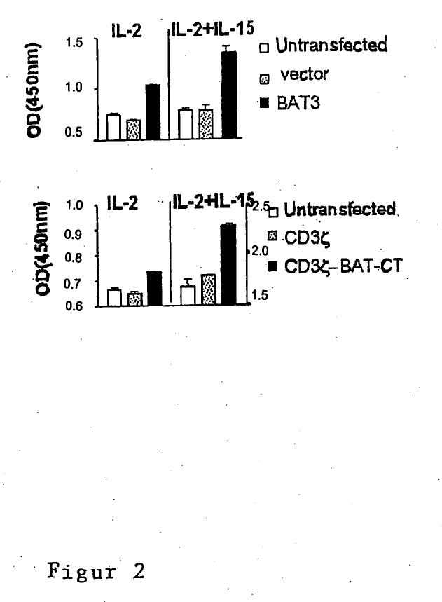

Another example focuses on the diagnostics related to these diseases and their therapy through the detection of BAT3 on the cells and in the serum. This may be achieved by antibody-based techniques, for example ELISA, FIG. 2), but also by means of other specific detection methods.

Object of the invention is therefore an agent for the therapy of patients suffering from multiple myeloma, said agent containing a physiologically effective amount of the protein BAT3/antiCD138 or BAT3 and derivatives of BAT3 or BAT3-specific antibodies as active substance in a pharmaceutically acceptable carrier material. Further possible areas of use are malignant diseases and immunologic diseases (allergies, autoimmune related diseases).

The agent according to the invention contains the anti-tumor immune response modulating protein BAT3 and derivatives of BAT3, BAT3/antiCD138fusion constructs or BAT3 specific antibodies as active substance for the therapy and diagnosis of malignant diseases and for modulating the immune system.

The inventive agent for the treatment of malignant diseases contains the active substance in a customary carrier material which is pharmaceutically acceptable and compatible. It may be expedient to administer the active substance in a systemic manner as is known, for example, in the interferon therapy of multiple sclerosis or for the treatment of diabetes.

Moreover, an ex-vivo application in the framework of cellular immunotherapies is another possible field of use.

It is to be understood that the protein or the encoding DNA and/or RNA or the specific BAT3 antibodies can be stabilized for storage purposes and to prevent premature losses of effectiveness. Such stabilization may be achieved by the admixture of customary additives such as buffering substances, salts of other proteins as well as DNA and RNA. Examples here are albumin, herring sperm, DNA, tRNA and detergents such as Triton, alkali and alkaline earth ions and the like. Storing the agent and/or active substance in dried or quick-frozen form or, after quick freezing, in liquid nitrogen may also be expedient.

The inventive agent may of course contain the active substance in modified form, i.e. as protein in which individual amino acids have been substituted or are missing. Prerequisite is that the active substance modified in this way still has the effect required for the stimulation of the immune effector cells in the treatment of patients suffering from malignant diseases. Reasons for such modifications may be aimed at stabilizing the active substance, improving the effect or effect spectrum, or may be reasons related to technical production and formulation issues. Conceivable in this context are mutation and fusion after chemical or genetical methods and fusions with N- and C-terminal proteins or peptides. This may lead, for example, to an improvement of the half-life of the protein. For reasons of better purification by means of affinity chromatography histidine tags or GST fusions may be resorted to, for example. Due to phosphorylation or glycosilation on suitable remnants modifications may be brought about that prevent degradation by naturally occurring proteases.

To stabilize the protein it may be expedient to make modifications, for instance on DNA level, to eliminate, for example, points of restriction, chemical instability or attacking possibilities for nucleases.

Adopting standard methods the protein may be obtained in a customary manner after cloning of the gene into suitable vectors recombinantly from bacteria or eukaryonts.

The agent according to the invention for treatment of malignant diseases by activating the immune effector cells contains the active substance in a carrier material which is pharmaceutically acceptable and may, on the one hand, consist of several customary constituents. On the other hand, the carrier material expediently contains a transfer medium suitable for the active substance. The agent is primarily intended for a systemic administration and the ex-vivo therapy in the framework of cellular immunotherapies.

Same as a recombinant BAT3 protein or BAT3 fusion protein or BAT3 specific antibodies the use of the BAT3 cDNA for an overexpression of BAT3 in tumor cells after viral and non-viral methods of gene transfer is object of the invention because BAT3 overexpressing tumor cells are suited, through a stimulation of NCR receptors, to support an anti-tumor immune response mediated by NK cells.

The invention is explained in more detail by the following examples wherein the effectiveness of BAT3 ligand is proven experimentally. For further elucidation of the examples and additional examples refer to the attached manuscript.

### EXAMPLES

### 1. The BAT3 Overexpression in Tumor Cells Leads to Activation of NK Cells:

### Increased Cytotoxicity Against Tumor Cells

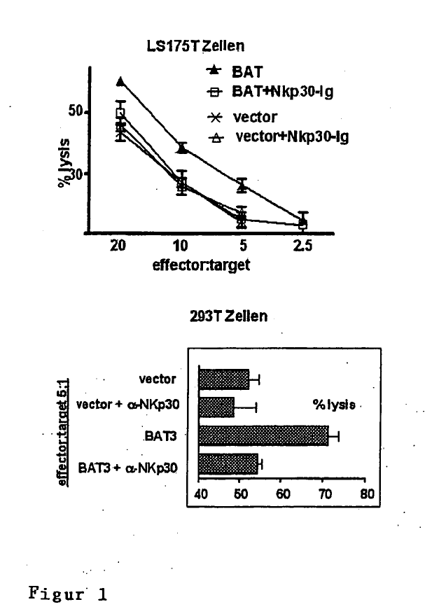

NK cells isolated from the blood of healthy donors were used as effector cells in europium release assays. In this process the target cells (human tumor cell lines) are marked with europium and incubated with NK cells at the effector-to-target ratios indicated. Subsequently, the amount of liberated europium in the supernatant is determined which is indicative of the NK cell mediated cell lysis. The value pertinent to a 100% lysis is arrived at after incubation of the cells with a detergent causing lysis to be effected. Additionally, spontaneous lysis is taken into account which is determined in batches without NK cells.
(A) For the europium release assay the NK cells (effector) were incubated at the ratios indicated with the human colon carcinoma line LS175T which was transfected with an expression construct for BAT3 (BAT3) or for comparison to the expression plasmid (vector). For all measuring points an increased lysis of the BAT3 expressing cells was found which is mediated via NKp30 because a recombinant NKp30 receptor inhibits this effect (BAT3+NKp30-lg).
(B) For the europium release assay the NK cells (effector) were incubated at a ratio of 5:1 with the human kidney carcinoma line 293T which was transfected with an expression construct for BAT3 (BAT3) or for comparison to the expression plasmid (vector). An increased lysis of the BAT3expressing cells was found which is mediated via NKp30 because the effect is inhibited by the addition of a masking NKp30 antibody (BAT3+α-NKp30). (Refer to FIG. 1)

### 2. The BAT3 Overexpression in Tumor Cells Leads to Activation of NK Cells:

### Stimulation of the Inteferon-γ Secretion

Expression vectors for BAT3 (BAT3) or for a membrane-bound BAT3 derivative (CD3ζ-BAT3-CT) respectively control vectors without BAT3 cDNA were introduced into LS175T cells. After 36 hours the turmor cells were mixed with primary NK cells stemming from the peripheral blood of healthy donors (see above) at a 1:1 ratio and incubated for another 48 hours. Prior to this the NK cells were stimulated overnight with interleukin 2 (10U) solely or in combination to with interleukin 15 (10 ng ml⁻¹). To determine the interferon g secretion of the NK cells interferon-γ was determined in the supernatants using an IFN-\tilde{γ} ELISA kit (human interferon-γELISA kit (R&D Systems). The overexpression of BAT3 (upper part of figure) dispensed by the tumor cells into the supernatant (evident from the Western Blot, see attached manuscript) and also of a membrane-bound variant (lower part of figure) stimulates the interferon g secretion of the NK cells. (Refer to FIG. 2)

### 3. BAT3is Essential for the Detection and Elimination of Tumors (Multiple Myeloma):

### In-Vivo Experiment (Mouse Xenograft Model)

Human multiple myeloma cells (line RPM18226) were injected into nude mice. This led to the formation of subcutaneous tumors in 8 of 10 mice. The tumor volume on day 13 and day 20 has been indicated (circles). If the mice are additionally given human peripheral blood lymphocytes (PBL) the tumor cells are eliminated and the formation of tumors is prevented in all cases. Moreover, the mice were given control antibodies (control: control antibodies) that do not impair the detection of tumor cells (black cross). However, if the mice are given BAT3-specific antibodies which deplete the endogenous BAT3 protein the detection and elimination of tumor cells is inhibited (yellow triangles, tumors in 6 of 10 mice).

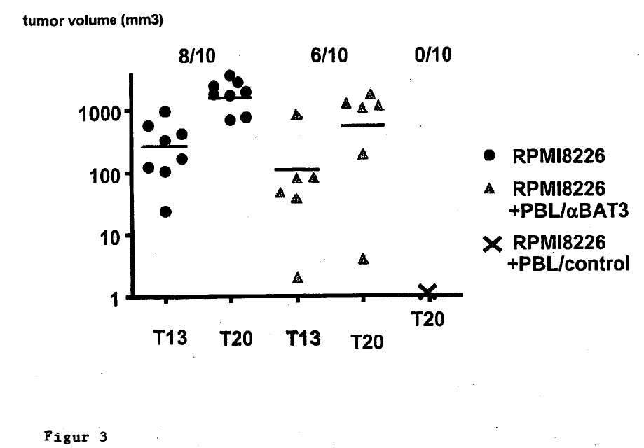

These experiments show that BAT3 in vivo is necessary for the detection and elimination of tumor cells. (Refer to FIG. 3)

1. Agent for treatment and/or diagnosis of tumor diseases, for example the multiple myeloma, through the activation of an anti-tumor immune response after stimulation of the NKp30 receptor and the natural cytotoxicity receptors, characterized in that said agent contains in an acceptable carrier material a physiologically effective amount of the BAT3 protein and/or BAT3/antiCD138 and/or a BAT3 fragment and/or BAT3-specific antibodies.

2. Agent according to claim 1, characterized in that the carrier material contains a transfer medium transporting the active substance.

3. Agent according to claim 1, characterized in that it is provided and applied in the form of an injection formulation.

4. Agent according to claim 1, characterized in that it is provided and applied in the form of a formulation for an ex-vivo application in the framework of a cellular immunotherapy for tumor patients.

5. Agent according to claim 1 characterized in that it is used for the therapy of CD138 expressing tumors, such as for example colon, lung or prostate carcinoma.

6. Agent according to claim 1 characterized in that said agent is used after the replacement of the antiCD138component with an antibody fragment by an optional tumor antigen for the therapy of tumors expressing this antigen.

7. Use of the BAT3 protein as active substance for the production of an agent according to claim 1 for the treatment of malignant diseases.

8. Use of the protein BAT3/antiCD138 as active substance for the production of an agent according to claim 1 for the treatment of malignant diseases.

9. Use of BAT3-specific antibody fragments as active substance for the production of an agent according to claim 1 for the treatment of malignant diseases.

10. Use of the BAT3 DNA and/or RNA for the production of BAT3/antiCD138-analogous constructs in which the CD138 bonding component is fused with an antibody fragment to any tumor antigen as active substance for the production of an agent according to claim 1 for the treatment of malignant diseases expressing the respective tumor antigen.

11. Use of the BAT3 cDNA for the in-vivo or ex-vivo overexpression of BAT3 in tumor cells after viral or non-viral gene transfer according to the state of the art for the immunotherapy of malignant diseases.
